(19) United States
(12) Patent Application Publication
(10) Pub. No.: US 2014/0186224 A1
(43) Pub. Date: Jul. 3, 2014

(54) BIODEGRADABLE ODOR REMOVING ARTICLE AND SYSTEM

(71) Applicant: HalolaH LLC, Weatogue, CT (US)

(72) Inventor: JulieAnn Derby Krans, Simsbury, CT (US)

(73) Assignee: HalolaH LLC, Weatogue, CT (US)

(21) Appl. No.: 14/145,909

(22) Filed: Dec. 31, 2013

Related U.S. Application Data
(60) Provisional application No. 61/848,265, filed on Dec. 31, 2012.

(51) Int. Cl. A61L 9/014

(52) U.S. Cl. CPC A61L 9/014
USPC 422/120

(57) ABSTRACT

A biodegradable odor and moisture absorbing article and system includes a biodegradable odor removing agent insert formed as a pouch for use removing odors and moisture from articles such as a bag, enclosure, or environment surrounding the person. The biodegradable pouch includes activated bamboo charcoal enclosed with a 100% breathable fabric (linen or hemp. The system includes an inner-facing layer of a dry touch breathable material such as fabric that wicks moisture into the pouch and an outer layer comprising a waterproof material or barrier (e.g. 100% vinyl backed polyester backing) configured to face the moisture such as a bag, enclosure, or environment surrounding the person. The article eliminates odor by the adsorption process when the pouch is inserted in the bag, enclosure, or environment surrounding the person.

The present invention claims the benefit of U.S. provisional patent application Ser. No. 61/848,265, filed Dec. 31, 2012.

### FIELD OF THE INVENTION

The present invention generally relates to containers and articles of clothing configured with an odor adsorption system to absorb wetness and odors emanation from a person, clothing or in the environment surrounding the person and, more particularly, to an odor adsorption system that includes at least one odor removing agent insert configured to be included in a pouch, bag or an article of clothing where the odor removing agent absorbs odors and moisture from the article. Additional aspects of the present invention are directed to methods of using the system.

### BACKGROUND OF THE INVENTION

Consumers are conscious about odors in their car, yoga or gym bags in their daily lives. After a heavy workout sweaty clothes & shoes are often put in bags for hours causing unpleasant odors. For example, sweaty clothes placed in an enclosure of a bag, car, locker or other stored area will start to smell within a short period of time and odors will increase proportionally, for a period of time, on the duration of time (period) left in an enclosed area. Other disadvantages of leaving sweaty clothing high humidity environments for extended durations include mold, mildew, or other biological growth on the clothing.

Additionally, consumers have become more conscious about their energy and water usage in their daily lives. Depending on the amount of clothing, it may not make sense to do a full load of laundry and small loads of laundry can waste energy and water. As a result, consumers can use of the same workout clothes over from days to weeks. Leaving such sweaty clothing in a yoga bag, gym bag or other plastic bag repeatedly may lead to the clothes retaining the odor after they are cleaned. Bags are also likely to be exposed to and retain these odors during periods of nonuse. Still also the car, locker or other stored area also is likely to be exposed to and retain these odors during periods of nonuse. As a result, a useful scent absorbing article and system with an insert of an improved lifespan, requiring replacement every 2 or more years, would overcome disadvantages in the prior art.

Previous attempted solutions include scented inserts to a bag and for an enclosure area; however, these have disadvantages a short duration of effectiveness whereby the useful scent duration of a cake insert is a month or so. Moreover, these products merely cover or otherwise mask the odor and do not remove the odor. Many odor covering, instead of odor removing, products exist yet these do not remove the odor. Encapsulating or using individual plastic bags to store the wet or otherwise sweaty clothing can be used; however, again, plastic bags have disadvantages as these do not remove the odor, can permanently impart clothing to the fabric of the clothing, and add to waste and, typically, are not biodegradable. As a result, a useful scent absorbing article and system that is biodegradable and removes odor and wetness is desired that would overcome the disadvantages in the prior art

A continuing need exists for ways to absorb odor emanation from closed environments such as a car or room, yoga or gym bag or clothing, while avoiding the waste associated with excessive use of water and energy by washing. Washing also reduces the life of the typical workout garment and using this solution may prolong the life of your workout clothes. Moreover, use of activated charcoal has failed because of the limited absorbing lifespan of conventional activated charcoal and that it is essentially non-renewable. A continuing need exists for a biodegradable insert article and system that has improved odor and water absorbing with an extended duration without the disadvantages of the prior art where odor removing inserts are frequently replaced to maintain a desired level of odor absorbing performance.

### SUMMARY OF THE INVENTION

It is an object of the present invention to provide a biodegradable odor removing article and system for use by a person to remove odors from articles such as a bag, enclosure, or environment surrounding the person such as the enclosed space of a car, yoga studio and the like that has the odor and moisture that will be absorbed by the system in various embodiments.

Embodiments of the present invention include an odor absorbing article of a pouch, comprising an odor removing agent insert; a core layer enclosing the odor removing agent insert; a layer of breathable material; and a layer comprised of waterproof material forming a waterproof barrier.

Another embodiment of the present invention is directed to a biodegradable moisture and odor absorbing system comprising an odor removing agent insert configured from active bamboo charcoal, a core layer enclosing the odor removing agent insert, a layer of breathable material, and a layer comprised of waterproof material forming a waterproof barrier. The odor removing agent insert advantageously comprises activated bamboo charcoal material with a long-life and that can be re-activated by exposure to sunlight. The core layer enclosing the odor removing agent insert is advantageously biodegradable consisting of hemp or linen material.

Another embodiment of the present invention is directed to a container for the absorption of moisture and odor comprising an article formed as a pouch comprising an odor removing agent insert enclosed by a core layer, an inner layer comprised of breathable, wicking material, and outer layer comprised of waterproof material thereby forming a waterproof barrier. The container has a base with a compartment configured to insert one or more articles with the outer layer disposed to the open container and a plurality of side panels affixed to the base, the sides adapted to be foldable adjacent the base so as to form side walls of the container, each wall is configured with a fastener means so that the walls positioned adjacent each other can be fastened at a corner thereof. The container advantageously utilizes the biodegradable article to remove odors and moisture from a bag, enclosure, or environment surrounding the person.

Other features and benefits of the present invention that characterize each embodiment will be apparent upon reading the following detailed description and review of the associated drawings.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 illustrates the article and system in accordance with an embodiment of the present invention;

FIG. 2 is a cross-sectional view of the article and system depicted in FIG. 1 taken generally along the line 2-2;

FIG. 3 is a schematic of adsorbtion of odors to the odor removing agent;

FIG. 4 is a schematic view illustrating an odor molecule binding to carbon substrate of the odor removing agent;

FIG. 5 is a simplified illustration of a drawstring bag in accordance with embodiments of the invention;

FIG. 6 is a simplified illustration of a duffel or gym bag in accordance with embodiments of the invention;

FIG. 7 is a simplified illustration of a yoga bag in accordance with embodiments of the invention;

FIG. 8 is a schematic illustration of a disassemble-able two- to three-dimensional shoebox in accordance with embodiments of the invention; and

FIG. 9 is a schematic illustration of the shoebox in accordance with embodiments of the invention

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “pouch” refers to an insert or other enclosed article in a pouch format with a biodegradable water and odor absorbing insert.

As used herein the term “biodegradable” refers to materials or substances where decomposition of biodegradable materials or substances, by either biological or abiotic steps, is effectuated by chemical dissolution of materials or substances by bacteria or other biological means.

As used herein the term “adsorption” refers to the adsorption process of intermolecular attraction (Vander Waals forces) of gas or vapor molecules to a magnetized surface (i.e. activated carbon via Bamboo charcoal). The attraction of the two molecules then modifies the bamboo charcoal structure by forming a bond with the odor compound. Due to the relatively weak forces involved, physical adsorption is (essentially) totally reversible. Adsorption is regarded as a surface phenomenon and a removal capacity of specific adsorbent is directly related to its total surface area.

As used herein “removing”, “absorb”, “absorbing” or “absorption” will be used to describe both adsorbing and absorbing functions.

As used herein “bag”, “enclosure,” or “environment surrounding the person” refers to the enclosed space housing the odor and moisture that will be absorbed by the system 100 of the present invention. For example, the pouch 101 can be inserted into air purifying bags, made of linen/hemp. These bags absorb unpleasant odors and dehumidify the air For example, the environment surrounding the person in an enclosed space of an automobile or other cabin, the system 100 can be configured for applications include using a 200 gram size pouch 101 to cover approximately 90 square feet in an automobile; and using a 300 gram pouch 101 to cover 135 square feet of a typical Sport Utility Vehicle (SUV). It has been shown that odors can be removed completely in day(s). Moreover, use of the activated bamboo charcoal is advantageously safe to use around pets and children in bags or pouch in compartments where inadvertent digestion could occur.

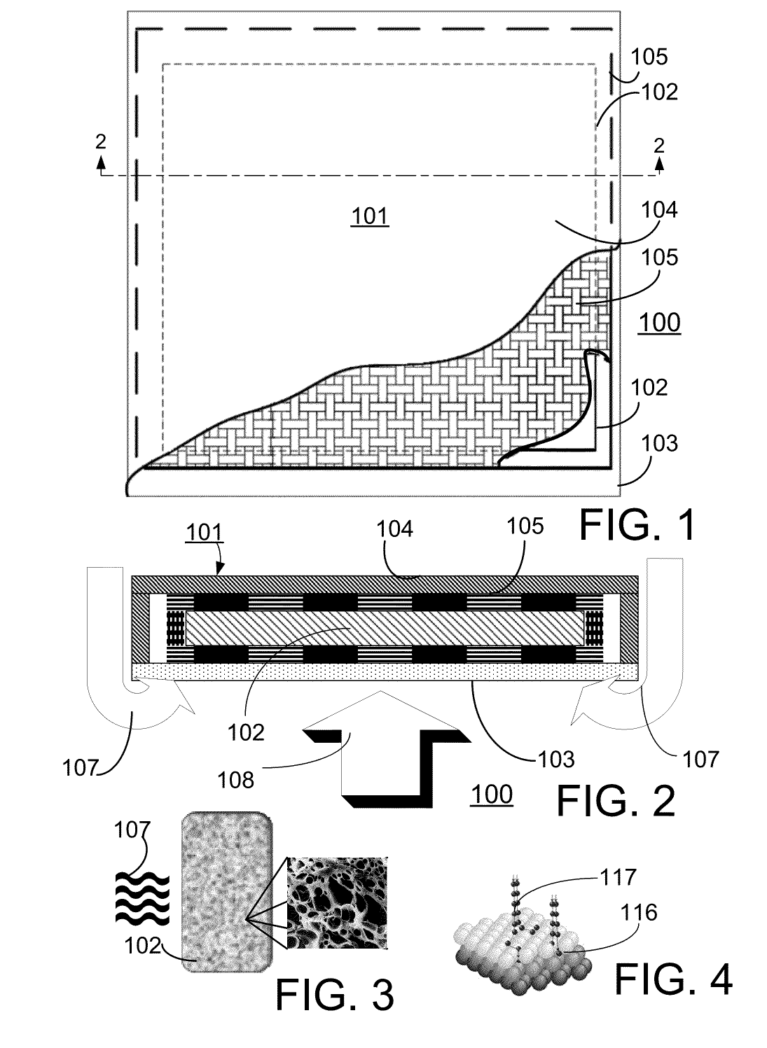

As is illustrated in FIGS. 1 through 9, one embodiment of the portable, biodegradable odor removing article and system is described and generally as element 100. Accordingly, the system 100 can be a pouch 101 of a multi-layer construction for use by a person to reduce odor emanation from clothing and confined spaces into an environment. FIG. 1 illustrates the layers of the pouch 101 in conjunction with a cross-sectional view of the system 100 depicted in FIG. 2 taken generally along line 2-2 in FIG. 1 in accordance with embodiments of the invention.

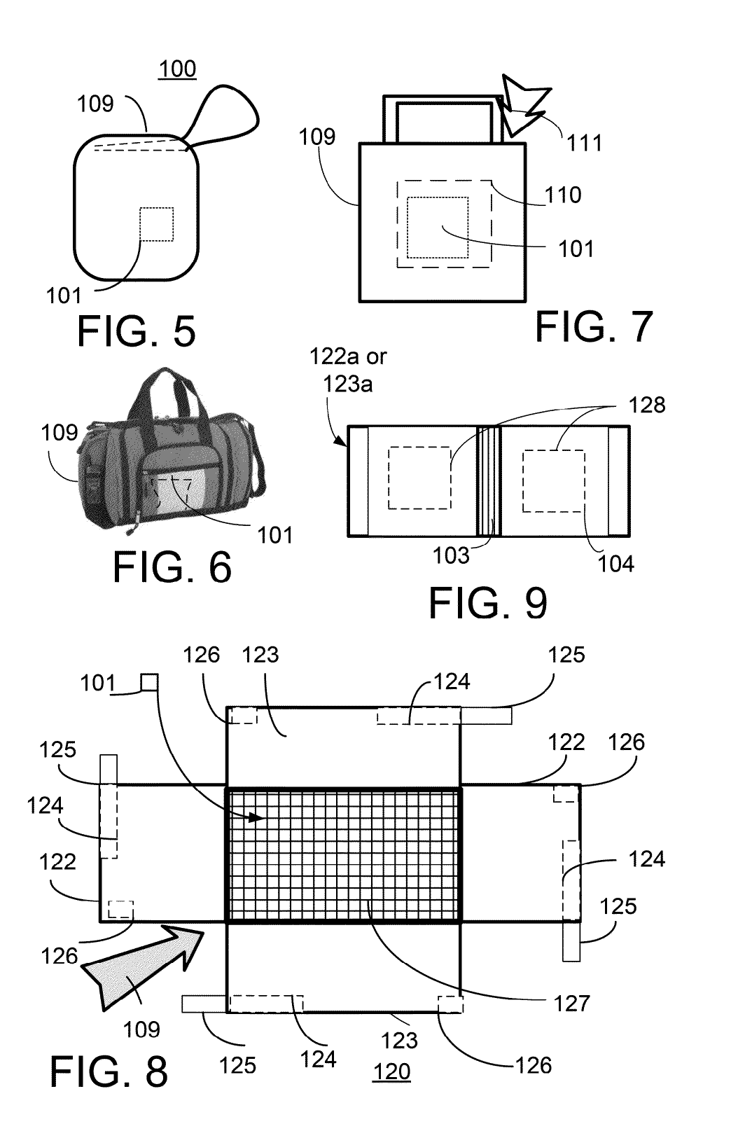

The system 100 includes can include a bag 109 such as, for example, a drawstring bag 109 for laundry or the like as shown in FIG. 5, a yoga bag as is shown in FIG. 6 or a duffle or gym bag as shown in FIG. 7 and a shoebox 120 as shown in FIG. 8. The system 100 includes can include an article of clothing 111

Referring to FIGS. 1 and 2, the system 100 includes an article of a pouch 101 configured with an odor removing agent insert 102, an inner layer 103, an outer layer 104 and a core layer 105. The odor removing agent insert 102 can be activated charcoal and is enclosed by the core layer 105. The inner layer 103 can be made from suitable wicking fabric, such as dry touch fabric, that functions to wick moisture into the pouch 101. The outer layer 104 can be made from suitable waterproof fabrics and materials functioning to create a waterproof barrier, for example, 100% vinyl backed polyester backing.

In operation, as is shown in FIGS. 1 and 2, the outer layer 104 is arranged away from the clothing or ambient air so as to allow odor 107 and moisture 108 to move into the pouch and be absorbed. The insert 102 actively removes moisture from clothing that passes through the inner layer 103 and core layer 105 to the active bamboo charcoal of the odor removing agent insert 102. The pouch 101 is configured to adsorb or absorb odors emanating from the bag 109 or clothing 111 or the environment surrounding the person.

Additionally, the system 100 includes at least one odor removing agent insert 102 that is configured with a core layer 105 to substantially cover or otherwise enclose the odor removing agent insert 102. The odor removing agent insert 102 can be configured and manufactured from 100% biodegradable Bamboo Charcoal (BC). The core layer 105 can be configured to enclose the bamboo charcoal and can be manufactured from 100% breathable, biodegradable fabric such as Linen or Hemp.

The odor removing agent insert 102 can be configured and manufactured from 100% biodegradable activated Bamboo Charcoal. Activated Bamboo Charcoal has suitable characteristics to provide adsorption of water and odors. Bamboo Charcoal is produced by incomplete combustion of moso-bamboo at high temperature and nitrogen atmosphere. The adsorption kinetics and thermodynamics of nitrogen-heterocyclic compounds (NHCs), pyridine, indole and quinoline. Thermodynamic analysis indicates that the adsorption is spontaneous and endothermic. Adsorption site energy analysis illustrates a distribution of adsorption energy, which indicates the heterogeneous sites on BC for NHC adsorption. Furthermore, spent BC with NHC adsorption can be effectively regenerated thereby allowing for reactivation of the odor and water absorption. The organic molecules actually meet the carbon-filtering layer (internal lining of the interior pouch) to remove smell.

Referring to FIGS. 3 and 4, the present invention utilizes the main attributes of activated carbon are an extremely high surface area (up to 1400 square meters per gram), a highly irregular pores structure with pore sizes ranging from 5 to 500 Angstroms and a non-polar chemistry which enables adsorption of a wide range of chemicals preferentially to moisture. For example, tests show that 1 gram of activated carbon has the surface area of more than two tennis courts. As shown in FIG. 3, at a microscopic level (electronic microscope magnified photo of an activated carbon pore structure), the carbon 116 forms layers that may only be a few nanometers apart. This makes activated carbon 116 ideal for absorption of chemicals such as ammonia 117 as the sponge-like surface area blocks the release of odor molecules as shown in FIG. 4.

As shown in Example 1, a nitrogen-heterocyclic A compounds contain this ring structure has an intermolecular attraction to an activated carbon structure B is

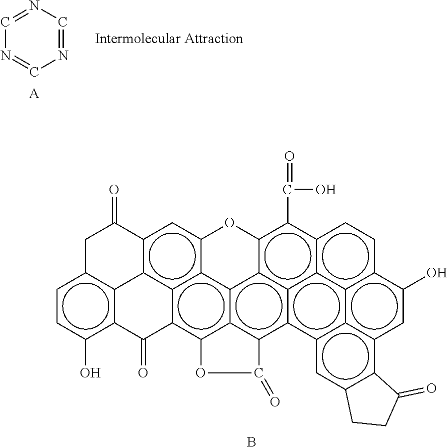

### EXAMPLE 1

Finally, it has been discovered that bamboo activated charcoal has rejuvenation properties. Bamboo charcoal naturally rejuvenates when the bags are placed in sunlight once a month for a minimum of 1 hour. When placed in the sun, the charcoal releases and disintegrates the pollutants to naturally rejuvenate (i.e. breaking the bonds formed to remove the original odor). Moreover, a pouch 101 can be dried in the sun, and flipped for other side, so the sun can fully rejuvenate the charcoal, thereby allowing advantageously cost effective reuse of the pouch 101 and the charcoal filled insert 102 for up to two years. At end of useful life, biodegradable disposal of the pouch 101 can advantageously occur directly to the earth, or ground and poured into the soil around plants to fertilize and help retain moisture, as the as the bamboo charcoal of the odor removing agent insert 102 and the hemp or linen of the core layer 105 can biodegrade.

Referring to FIGS. 5 through 7, in accordance with one embodiment of the invention, the pouch 101 containing an odor absorbing agent insert 102 is configured for removable attachment to an article of a bag 109. For instance, the pouch 101 can be attached directly to the drawstring bag 109 using one or more conventional fasteners such as stitching as is shown in FIG. 5, or alternatively, the pouch 101 can be inserted into vented inner pocket 110 as is shown in FIG. 7 of the yoga bag. Examples of suitable fasteners include stitching, hook and loop fasteners (e.g., Velcro®, buttons, zippers, or other suitable fasteners), which allow for the removal of the odor removing agent insert 102 without damaging the article 109. For example, a plurality of fasteners can be provided each having one portion attached to the odor removing agent insert 102, and another portion attached to the layer 106 of the article 109.

Referring to FIG. 8 a two- to three-dimensional shoebox 120 design can be utilizes that incorporates pouch 101 inserts into the base so as to configured to adsorb or absorb odors emanating from clothing or articles disposed therein. Moreover, the shoebox 120 advantageously can be utilized to remove body odor in enclosed spaces such as, for example, the environment surrounding the person in the surrounding space of a yoga studio by placing the shoebox 120 in the room. The shoebox 120 can be formed from a generally planar surface having a base 121, at least one a short side panel 122 and long side panel 123.

As shown in FIG. 8, each of the short and long sides 122 and 123, respectively can have a fastener means 124 for making a general-shoebox-shape when the sides 122 and 123 are folded upwardly and the fastener means 124 secures the sides together. For example, the fastener means 124 can be a strap 125 of hook fabric configured to secure to a loop portion, whereby each of the hook strap and portion of loop fabric, or vice versa, is disposed on the outside of the short and long sides 122 and 123 to secure these sides together. The pouch 101 is disposed on a compartment 127 in the base 121 with the waterproof outer layer 104 facing upwardly or to the inside of the shoebox 120. The pouch 101 can be utilizes that incorporates pouch 101 inserts into the base so as to configured to adsorb or absorb odors emanating clothing 111 or the surrounding space. In operation, for example, an article of clothing or a drawstring bag 109 full of towels and clothing, shown in FIG. 5, can be placed into box of the enclosure formed by securing the sides of the shoebox 120.

### EXAMPLE 2

As shown in Table 1, the pouch 101 can be effective and efficient at eliminating odors and moisture in an enclosure, or environment surrounding the person. For example, the amount of activated bamboo charcoal needed to remove odors from

TABLE 1

| | Amount of Activated | |
|---|---|---|
| Location of Use | Bamboo Charcoal stored | Cube Size |
| (associated square feet of | in a Cube of pouch 101 | (Length × Width × |
| odor removal coverage) | (grams) | Height) |
| Car (90 ft²) | 200 g | 3″ × 3″ × 3″ |
| SUV (135 ft²) | 300 g | 3.5″ × 3.5″ × 3.5″ |
| Yoga Studio (300 ft²) | 700 g | 4.75″ × 4.75″ × 4.75″ |

In an alternative embodiment illustrated by FIGS. 8 and 9, the side panels 122a and 123a can be comprised of a construction with odor removing agent panel 128 and incorporated into the side panels of the two- to three-dimensional shoebox 120 design. The base 121 can be a predetermined dimension, for example, in this embodiment the base has dimensions of 5″×11″×1.5″ (depth). Similarly, each side portion can have a predetermined dimension, for example, the short side panel 122 can be dimensioned as 5″×7″×1.5″ and the long side panel 123 can be dimensioned 5″×11″×1.5″. The strap 125 can be dimensioned to 1″×4″×1″. The side panels 122 and 123 can have a suitable multi-layer construction have an the outer layer 104 facing the moisture to protect the active bamboo charcoal as well as to have the inner layer 103 arranged away from the clothing or ambient air so as to allow odor 107 and moisture 108 to move into the odor removing agent panel 128 and be absorbed. In operation, moisture is removed from clothing placed in the shoebox 120 as it passes through the inner layer 103 to the active bamboo charcoal of the odor removing agent panel 128.

While certain configurations of structures have been illustrated for the purposes of presenting the basic structures of the present invention, one of ordinary skill in the art will appreciate that other variations are possible which would still fall within the scope of the appended claims. For example, the pouch of the present invention can be formed in different shapes and geometries for a shoe application to reduce odor and remove wetness from sweaty shoes. The system 100 can be incorporated into an article of clothing 111 to eliminate moisture and body odor. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An odor absorbing article of a pouch, comprising:
an odor removing agent insert;
a core layer enclosing the odor removing agent insert;
a layer of breathable material; and
a layer comprised of waterproof material forming a waterproof barrier

2. The article of claim 1 wherein the odor removing agent insert is activated bamboo charcoal material.

3. The article of claim 2 wherein the odor removing agent insert is biodegradable.

4. The article of claim 1 wherein the core layer enclosing the odor removing agent insert is biodegradable and formed from the group of hemp or linen material.

5. An biodegradable moisture and odor absorbing system comprising:
an odor removing agent insert configured from active bamboo charcoal.
a core layer enclosing the odor removing agent insert;
a layer of breathable material; and
a layer comprised of waterproof material forming a waterproof barrier.

6. The article of claim 5 wherein the odor removing agent insert 102 is selected from active bamboo charcoal.

7. A container for the absorption of moisture and odor comprising:
an article formed as a pouch comprising an odor removing agent insert enclosed by a core layer, said article further including an inner layer comprised of wicking material and outer layer comprised of waterproof material forming a waterproof barrier
a base with a compartment configured to insert one or more articles with the outer layer disposed to the open container;
a plurality of walls affixed to the base, the sides adapted to be foldable adjacent the base so as to form side walls of the container, each wall is configured with a fastener means so that the walls positioned adjacent each other can be fastened at a corner thereof;
the container being characterized by:
the biodegradable article removing odors and moisture from a bag, enclosure, or environment surrounding the person.

8. The container of claim 5 wherein the container is a flexible bag.

9. The container of claim 5 wherein the odor removing agent insert is active bamboo charcoal.
